(19) United States
(12) Patent Application Publication
(10) Pub. No.: US 2005/0033030 A1
(43) Pub. Date: Feb. 10, 2005

(54) Humanised antibodies and uses thereof

(76) Inventors: Benny Kwan Ching Lo, Cambridge (GB); Laughton Charles Anthony, Nottingham (GB); Michael Rawlings Price, Nottingham (GB)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/433,349

(22) PCT Filed: Nov. 29, 2001
(86) PCT No.: PCT/GB01/05260

(30) Foreign Application Priority Data
Dec. 1, 2000 (GB) 0029360.5

(51) Int. Cl. A61K 39/395

(52) U.S. Cl. 530/388.15; 424/141.1

(57) ABSTRACT

A humanised antibody capable of binding to the MUC1 mucin antigen comprises a light chain and a heavy chain. The variable region of the light chain (V_{L}) comprising an amino acid sequence which is substantially homologous with the sequence of FIG. 1A and the variable region of the heavy chain (V_{H}) comprising an amino acid sequence which is substantially homologous with the sequence of FIG. 1B. The amino acid residue at position 46 on V_{L} is backmutated to arginine, and the amino acid residue at position 47 on V_{H} is backmutated to leucine. The humanised antibody has use in the diagnosis and/or treatment of cancer.

### INTRODUCTION

The invention relates to a humanised version of the murine C595 antibody, and to uses of the humanised antibody in the diagnosis, staging and treatment of cancers.

The MUC1 mucin is expressed by secretory epithelia. Its abberant glycosylation in tumours allows it to be exploited as a marker for antibody targeted diagnosis and therapy. The C595 murine monoclonal antibody targets the epitope Arg-Pro-Ala-Pro on the MUC1 protein core. It has been used both in-vitro and in-vivo in the diagnosis of breast and bladder cancer. A phase 1 clinical trial of the antibody as a radioimmunotherapeutic agent in bladder cancer by intravesical administration has recently been initiated. Its potential use as an intravenous diagnostic has been limited by its murine origin.

It is an object of the invention to overcome this problem.

### STATEMENTS OF INVENTION

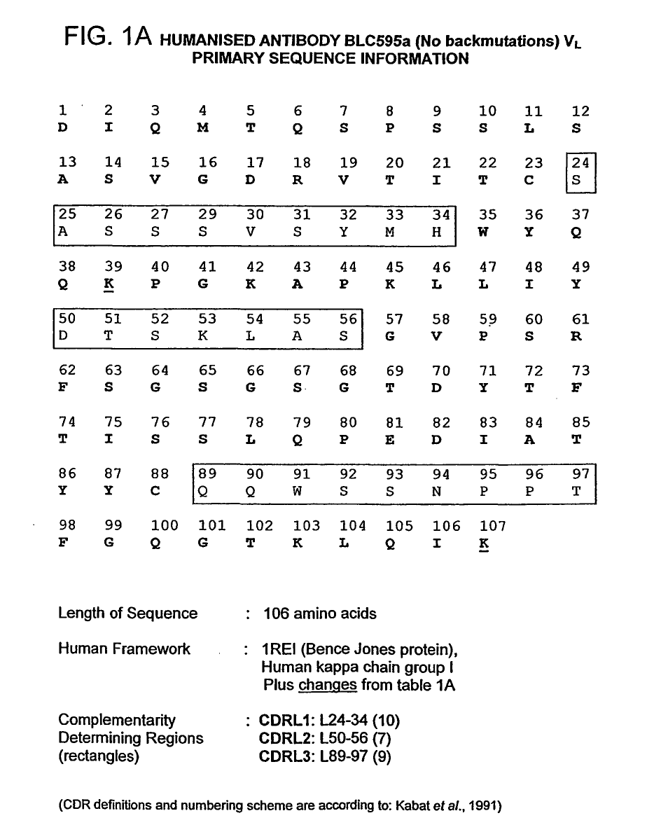

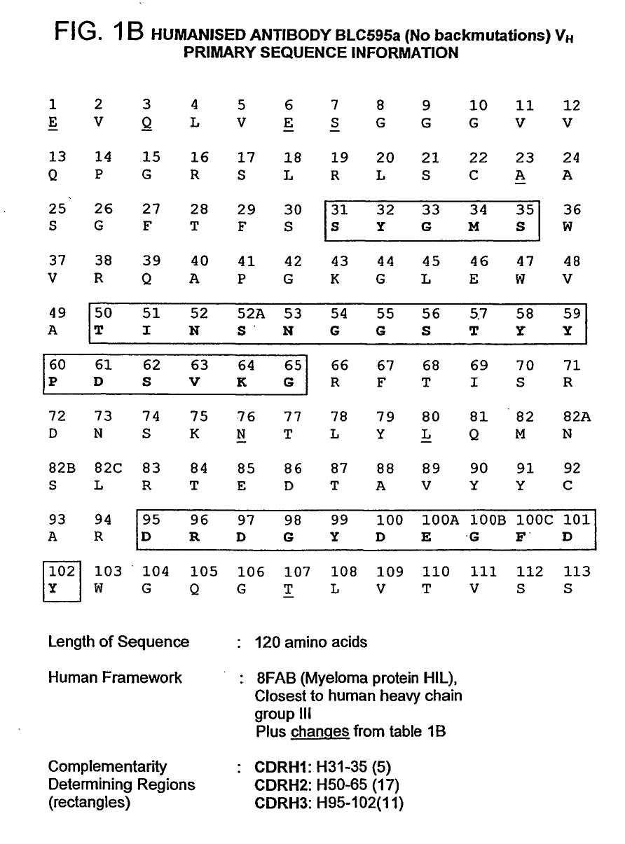

Accordingly, the invention provides a humanised antibody capable of binding to the MUC1 mucin antigen comprising a light chain and a heavy chain, the variable region of the light chain (V_{L}) comprising an amino acid sequence which is substantially homologous with the sequence of FIG. 1A, the variable region of the heavy chain (V_{H}) comprising an amino acid sequence which is substantially homologous with the sequence of FIG. 1B wherein the amino acid residue at position 46 on V_{L} is backmutated to arginine, and wherein the amino acid residue at position 47 on V_{H} is backmutated to leucine. The V_{L} domain is joined to the human immunoglobulin Kappa constant domain to form the complete light chain. Similarly, the V_{H} domain is joined to the human immunoglobulin gamma-1 constant domains to form the complete heavy chain.

In this specification the term “substantially homologous” should be understood as meaning that the degree of homology is sufficient to allow binding to the MUC1 mucin antigen when any of the various backmutation combinations of the invention are included. Thus, stated another way, the antibodies according to the invention comprise a light chain and a heavy chain, the V_{L} domain of the light chain comprising a framework region (FR) derived from the Bence Jones protein REI and complementarity-determining regions (CDR) derived from the murine C595 antibody, the FR including at least one backmutation at position 46 to arginine, the V_{H} domain of the heavy chain comprising a FR derived from myeloma protein HIL and CDR derived from murine C595 antibody, the FR including at least one backmutation at position 47 to leucine.

Typically, the V_{L} domain will have at least a 60%, preferably at least 70%, more preferably at least 80%, more preferably at least 90%, and most preferably at least 95% homology with the amino acid sequence of FIG. 1A

Similarly, the V_{H} domain will typically have at least a 60%, preferably at least 70%, more preferably at least 80%, more preferably at least 90%, and most preferably at least 95% homology with the amino acid sequence of FIG. 1B.

Preferably, the V_{L} domain will include further backmutations to improve binding affinity. In one embodiment of the invention the amino acid residue at position 4 of the V_{L} domain is backmutated to leucine.

Preferably, the amino acid residues at positions 4 and 1 of the V_{L} domain are backmutated to leucine and glutamine respectively. Ideally, the amino acid residues at positions 4, 1 and 47 on the V_{L} domain are backmutated to leucine, glutamine and tryptophan respectively. The combination of these three backmutations with the backmutation on residue 46 of the V_{L} domain has the effect of increasing the affinity of the humanised antibody for the antigen seven-fold. Suitably, the amino acid residues at positions 4, 1, 47 and 3 on the V_{L} domain are backmutated to leucine, glutamine, tryptophan and valine respectively. Typically, the amino acid residues at positions 4, 1, 47, 3, 40 and 70 on the V_{L} domain may be backmutated to leucine, glutamine, tryptophan, valine, serine and serine respectively.

In another embodiment of the invention, the amino acid residues at positions 4 and 47 on the V_{L} domain are backmutated to leucine and tryptophan. In a further embodiment of the invention the amino acid residue at position 47 on the V_{L} domain is backmutated to tryptophan. In a still further embodiment of the invention, the amino acid residues at positions 1, 3 and 4 on the V_{L} domain are backmutated to glutamine, valine and leucine.

The possible permutations for back mutations to the V_{L} domain according to the invention is summarised in Table 2A.

Preferably, the V_{H} domain will include further backmutations. Thus, for example, the backmutation of the amino acid residue at position 42 on the V_{H} domain to aspartic acid has been found to increase the binding affinity of the antibody two-fold. Furthermore, the backmutation of the amino acid residue at position 16 on the V_{H} domain to glycine has been demonstrated to reduce the non-specific binding of the antibody to other unrelated antigens. The possible backmutation permutations of the V_{H} domain according to the invention are summarised in Table 2B.

Most preferably, the humanised antibody comprises the backmutation indicated as BMLr in Table 2A and the backmutation indicated as BMHq in Table 2B.

The V_{L} domain according to the invention typically comprises a framework region (FR) and complementarity determining regions (CDR), wherein the FR region is derived from the Bence Jones protein REI, and wherein the CDR is obtained from the C595 antibody.

The V_{H} domain according to the invention typically comprises a framework region (FR) and complementarity determining regions (CDR), wherein the FR region is derived from the myeloma protein HIL, and wherein the CDR is obtained from the C595 antibody.

In a preferred embodiment of the invention, the humanised antibody according to the invention is conjugated to a radioactive isotope. Ideally, the radioactive isotope is selected from the group of Technetium-99m, Rhenium-188, Copper-67 and Indium-111.

The invention also relates to the use of a humanised antibody according to the invention in the diagnosis and/or treatment of cancer, in the intravesical diagnosis and/or therapy of bladder tumour and/or bladder cancer, in the intravenous diagnosis, staging and/or therapy of metastatic bladder cancer, and in the intravenous diagnosis and/or therapy of localised and/or metastatic cancers expressing the MUC1 mucin antigen, especially bladder, breast and ovarian cancers.

The invention also relates to a variable light chain domain (V_{L}) for a humanised antibody according to the invention comprising an amino acid sequence which has a sufficient degree of homology with the sequence of FIG. 1A to allow binding to the MUC1 mucin antigen when one of the backmutation combinations given in Table 2A is included.

The invention also relates to a variable heavy chain domain (V_{H}) for a humanised antibody according to the invention and comprising an amino acid sequence which has a sufficient degree of homology with the sequence of FIG. 1B to allow binding to the MUC₁ mucin antigen one of the backmutation combinations given in Table 2B is included.

The invention also relates to the use of the V_{L} domain and/or the V_{H} domain of the invention in the formation of a humanised antibody and/or an antibody binding fragment (e.g. single chain FV antibody, diabody, and other multivalent derivatives) which is capable of binding to the MUC1 mucin antigen.

The invention also seeks to provide a method for the treatment or diagnosis of cancer, comprising administering an effective amount of a humanised antibody according to the invention to a patient.

The invention also provides a humanised antibody according to the invention for use in the manufacture of a medicament for the treatment or diagnosis of cancer.

### DETAILED DESCRIPTION OF THE INVENTION

Preparation of Human Framework Regions for CDR Grafting:

The framework regions (FRs) from the Bence-Jones protein REI [V_{L}, Protein databank [PDB] access code: 1REI, Kabat subgroup (Kabat et al., 1991): human kappa I] and the myeloma protein HIL (V_{H}, PDB access code: 8FAB, Kabat subgroup: human heavy III) were used as acceptor FRs for the CDRs from C595 in CDR grafting. A number of amino acid residues in these FRs were substituted by the consensus residue at those positions within the corresponding subgroup because of their relatively low occurrence in the subgroups and are therefore likely to have arisen from idiosyncratic mutations (table 1). These substitutions ensure that the human FRs represents human immunoglobulin sequences as a whole, rather than an individual sequence containing unnecessary mutations (which may only be useful for that particular antibody). All substituted residues are already present in the original murine C595 sequence and therefore such substitutions should not be detrimental to antigen binding. Tyr-71(V_{L}) was not substituted because it is positioned in the Vernier zone (Foote and Winter, 1992) of C595 V_{L} and may have important interactions with the CDRs.

TABLE 1
Residues in the FRs of (a) 1rei and (b) 8fab which deviate
from the consensus sequence within their Kabat subgroups.

| | | |
|---|---|---|
| | | Substitution by consensus |
| | Occurrence | (first letter = original residue |
| | in Kabat | number = Kabat residue number |
| Residue | subgroup (%) | last letter = consensus substitution) |

(A) 1rei (V_{L}) - human subgroup kappa I

| | | |
|---|---|---|
| Thr-39 | 3 | T39K |
| Tyr-71 | 3 | No - Vernier zone residue |
| Phe-73 | 26 | — |
| Ile-83 | 21 | — |
| Leu-104 | 24 | — |
| Thr-107 | 5 | T107K |

(B) 8fab (V_{H}) - human subgroup heavy III

| | | |
|---|---|---|
| PCA*-1 | 12 | PCA1E* |
| Lys-3 | 2 | K3Q |
| Gln-6 | 6 | Q6E |
| Ala-7 | 2 | A7S |
| Val-11 | 25 | — |
| Arg-16 | 28 | — |
| Ile-23 | 2 | I23A |
| Ala-49 | 30 | — |
| Arg-76 | 2 | R76N |
| Met-80 | 3 | M80L |
| Thr-84 | 10 | — |
| Val-107 | 2 | V107T |

*PCA = pyrollidone carboxylic acid

CDR Grafting:

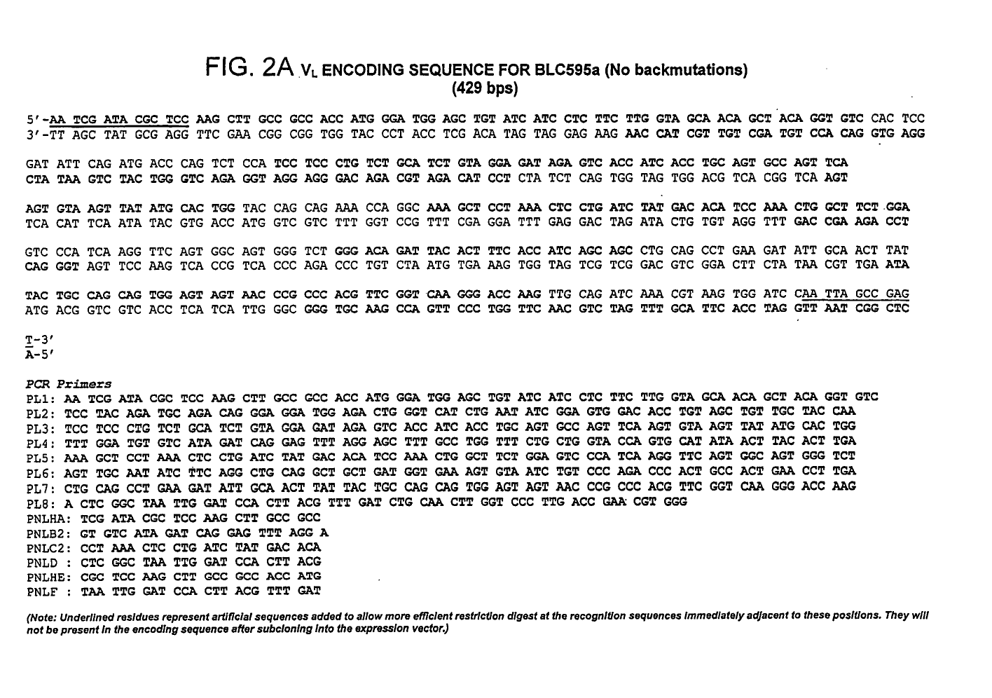

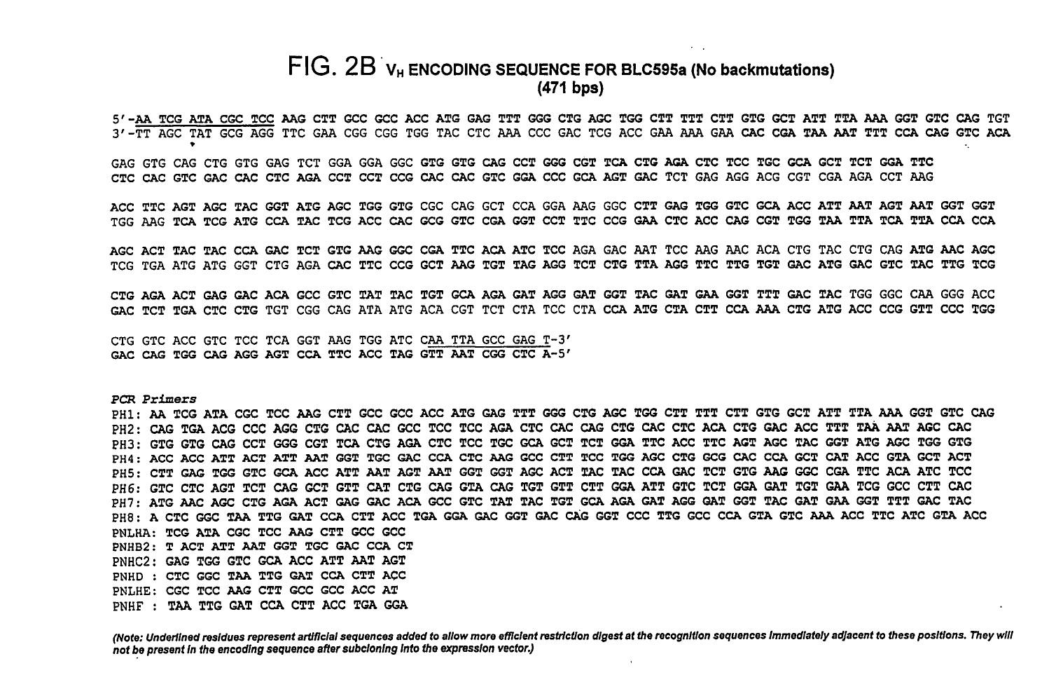

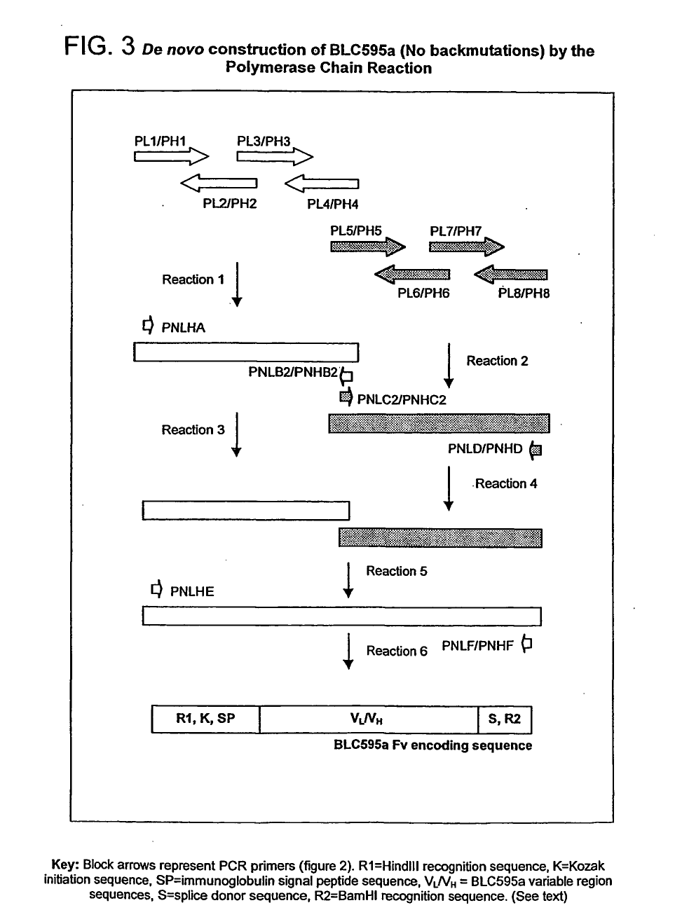

The finalised FRs were joined to CDRs from C595 to form the sequence BLC595a. The complete amino acid sequence of the BLC595a variable region is shown in FIG. 1. The DNA sequence for BLC595a was then deduced according to common codon usage for immunoglobulins (Kabat et al, 1991). To this DNA sequence, a cassette containing the recognition sequence for the restriction enzyme HindIII [(MG:CTT) (other suitable restriction enzyme recognition sequences may also be used for subcloning into expression vectors)], the Kozak initiation sequence (Kozak, 1987) and an immunoglobulin signal peptide sequence from the antibody sharing the highest sequence homology with the corresponding humanised V_{L} and V_{H} domains (i.e. BLC595 V_{L} and V_{H}) published in the Kabat database (Kabat et al., 1991) were added upstream. Also, a splice donor site (Bendig and Jones, 1996; optional depending on the expression vectors used) and the recognition sequence for the restriction enzyme BamHI [(GGA:CTT), or other appropriate restriction enzyme recognition sequence] were added downstream to this sequence. This whole sequence (i.e. HindIII-Kozak-signal-BLC595 V_{L}/V_{H}-splice donor-BamHI; to be referred to as “the encoding sequence”) for each of V_{L} and V_{H} was then analysed for the presence of internal splice donor and restriction sites (e.g. BamHI/HindM) with the Genetics Computer Group (GCG) Wisconsin Package v.9.0. The complete DNA encoding sequences for BLC595a V_{L} and V_{H} are shown in FIG. 2.

The encoding sequences were synthesised de novo by the polymerase chain reaction (PCR). Eight overlapping oligonucleotide primers (each of around 80-nucleotide in length; FIG. 2) were synthesised to cover each of the V_{L} and V_{H} encoding sequences for BLC595a in a series of PCRs (Bendig and Jones, 1997; FIG. 3). The PCR products representing full length V_{L} and V_{H} were cloned and their sequences confirmed to yield the CDR-grafted sequence BLC595a.

PCR for BLC595a construction (Referring to FIG. 3)
1) Reactions 1 and 2:

| | |
|---|---|
| 5 μL | Geneamp 10 × PCR buffer with 15 mM MgCl₂ (Perkin- |
| | Elmer) |
| 1 μL | 10 mM dNTP Mix (Sigma) |
| 12.5 pmol | each of PL/H1, 2, 3, 4 (reaction 1 - V_{L}/V_{H}) or PL/H5, 6, |
| | 7, 8 (reaction 2 V_{L}/V_{H}) |
| 2.5 units | AmpliTaq DNA polymerase (Perkin Elmer) + sufficient |
| | sterilised, deionised water to 50 μL |
| Conditions: | 1) 94° C. - 5 minutes (hot start) |
| | 2) 94° C. - 2 minutes) × 8 cycles |
| | 72° C. - 5 minutes) |
| | 3) 72° C. - 10 minutes |

2) Reactions 3, 4 and 6

| | |
|---|---|
| 5 μL | Geneamp 10x PCR buffer with 15 mM MgCl₂ (Perkin- |
| | Elmer) |
| 1 μL | 10 mM dNTP Mix (Sigma) |
| 5 μL | PCR product from reaction 1 (reaction 3, V_{L}/V_{H}), reaction |
| | 2 (reaction 4, V_{L}/V_{H}) or reaction 5 (reaction 6 - V_{L}/V_{H}) |
| 40 pmol | PNLHA and PNLB2 (reaction 3, V_{L}) |
| each | PNLHA and PNHB2 (reaction 3, V_{H}) |
| | PNLC2 and PNLD (reaction 4, V_{L}) |
| | PNHC2 and PNHD (reaction 4, V_{H}) |
| | PNLHE and PNLF (reaction 6, V_{L}) |
| | PNLHE and PNHF (reaction 6, V_{H}) |
| 2.5 units | AmpliTaq DNA polymerase (Perkin Elmer) + sufficient |
| | sterilised, deionised water to 50 μL |
| Conditions: | 1) 94° C. - 5 minutes (hot start) |
| | 2) 94° C. - 1.5 minutes) |
| | 64° C. - 1.5 minutes) × 20 cycles |
| | 72° C. - 2.5 minutes) |
| | 3) 72° C. - 10 minutes |

3) Reaction 5:

| | |
|---|---|
| 5 μL | Geneamp 10x PCR buffer with 15 mM MgCl₂ (Perkin- |
| | Elmer) |
| 1 μL | 10 mM dNTP Mix (Sigma) |
| 5 μL each | PCR products from reactions 3 and 4 (V_{L}/V_{H}) |
| 2.5 units | AmpliTaq DNA polymerase (Perkin Elmer) + sufficient |
| | sterilised, deionised water to 50 μL |
| Conditions: | 1) 94° C. - 5 minutes (hot start) |
| | 2) 94° C. - 2 minutes) × 8 cycles |
| | 72° C. - 5 minutes) |
| | 3) 72° C. - 10 minutes |

Introduction of Backmutations:

Backmutations are defined as the substitution of the amino acid residue at a position in the chosen human framework with the residue at the same position in the mouse antibody C595. These were introduced in an attempt to optimise the antigen binding ability of BLC595 after CDR grafting. Mutations were introduced by the method of overlap extension PCR (Higuchi et al. 1988). All mutants were cloned and sequenced prior to antibody expression. A number of backmutants of V_{L} and V_{H} were made that incorporated one or more such amino acid backmutations. The positions for backmutations were determined initially on the common framework positions known to affect CDR conformations [namely, the Vernier zone (Foote and Winter, 1992), V_{L}/V_{H} interface (Chothia et al., 1985), V_{L} N-terminal residues (Padlan, 1994) and putative O- and N-glycosylation syites (Bendig and Jones, 1997)]. These were exhausted before other backmutations were explored. In the case of BLC595, it was mainly the other backmutations, which were not obvious from previous publications, that led to a high level of restoration to specific MUC1 binding. Mutations in all the backmutants (represented by BMLx for V_{L} mutants and BMHx for V_{H} mutants) are shown in table 2 below.

TABLE 2
Mutations incorporated into the human frameworks. The first
letter of each backmutation indicates the original amino acid residue in the
human framework. The number indicates the amino acid position (Kabat
numbering system; Kabat et al, 1991). The last letter indicates the new amino
acid residue after backmutation.
(A) BLC595 V_{L} backmutants

| | Backmutations | | | | | | |
|---|---|---|---|---|---|---|---|
| Backmutant | D1Q | Q3V | M4L | P40S | L46R | L47W | D70S |
| BMLb | • | • | • | • | • | • | • |
| BMLc | | | • | | • | • | |
| BMLd | | | | | • | | |
| BMLg | • | • | • | | • | • | |
| BMLj | | | • | | • | | |
| BMLm | | | | | • | • | |
| BMLn | • | • | • | | • | | |
| BMLp | • | | • | | • | | |
| BMLq | | • | • | | • | | |
| BMLr | • | | • | | • | • | |

(B) BLC595 V_{H} backmutants:

| Back | | | | | | | | | | | | | | |
|---|---|---|---|---|---|---|---|---|---|---|---|---|---|---|
| mutant | V11L | R16G | R19K | A40T | G42D | G44R | W47L | S74A | N(82A)S | R83K | T84S | V89M | L108T | V109L |
| BMHb | | | | • | | | • | | • | | | | • | |
| BMHc | | | | | | | • | | | | | | | |
| BMHe | | | | | • | • | • | | | | | | | |
| BMHf | | | | | | | • | • | | | | | | |
| BMHg | | | | | | | • | | | | | • | | |
| BMHi | | | | | | | • | | | | | | • | • |
| BMHj | | | | | | | • | | | • | • | | | |
| BMHk | • | • | • | | | | • | | | | | | | |
| BMHm | | | | | • | | • | | | | | | | |
| BMHn | | | | | | • | • | | | | | | | |
| BMHp | • | | | | • | | • | | | | | | | |
| BMHq | | • | | | • | | • | | | | | | | |
| BMHr | | | • | | • | | • | | | | | | | |

Final BLC595 Sequence and Antibody Expression.

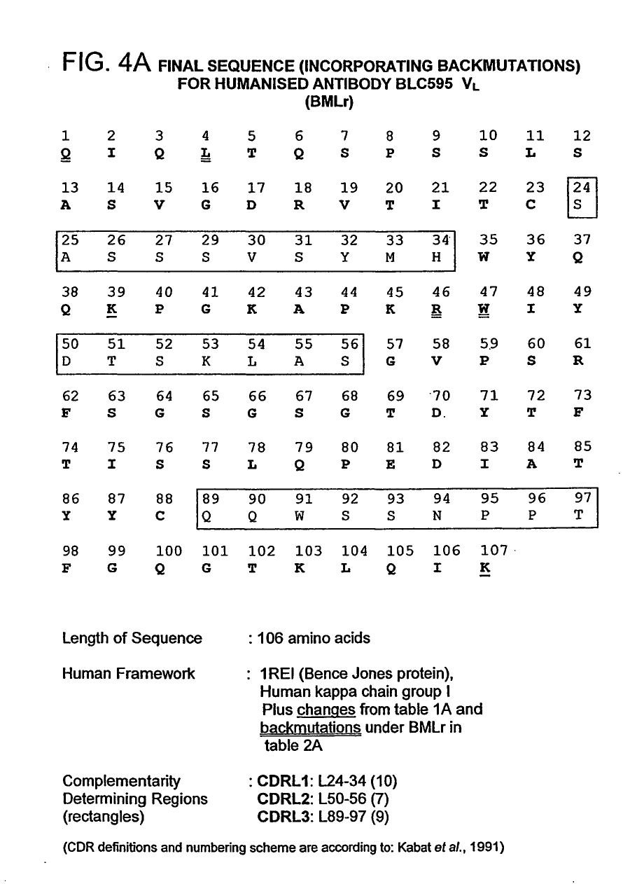

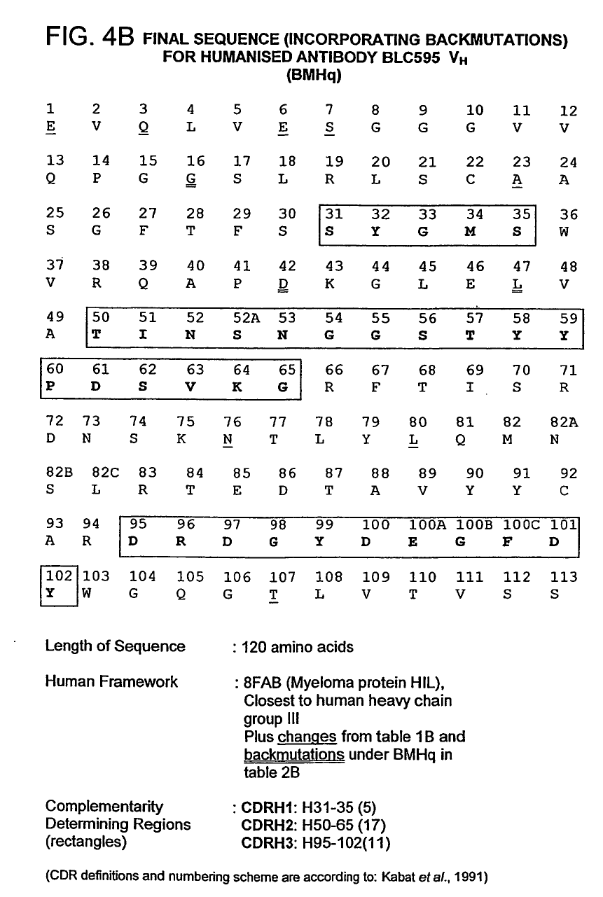

The final BLC595 variable region consists of the backmutants BMLr and BMHq. The complete amino acid sequences are shown in FIG. 4. The encoding sequences for BMLr and BMHq were excised from the cloning vector by appropriate restriction digests and were subcloned into expression vectors containing the human constant regions kappa and gamma-1 respectively for whole IgG expression (for example, pKN10—light chain; pG1D16/20—heavy chain—from Medical Research Council Technology). These BLC595 expression vectors (for example, 10 μg each of pKN10-BLC595 V_{L} and pG1D16/20—BLC595 V_{H}) were then co-transfected into 7×10⁶ COS-7 cells by electroporation at 1900V, 25 μF. Cells were then transferred to 8 mLs of pre-warmed medium (Dulbecco modified eagle medium supplemented with 10% (v/v) ultra low IgG-foetal bovine serum, 580 μg/ml L-glutamine and 50 Units/ml penicillin/50 μg/ml streptomycin). Antibodies were harvested in the medium 48-72 hours post transfection. Purified BLC595 was obtained by standard Sepharose-protein A affinity chromatography.

Methods for Radiolabelling of Antibodies

We envisage the use of ^{99m}Tc (or other gamma-emitting isotopes) as a diagnostic radionuclide and ¹⁸⁸Re (or other gamma- and beta-emitting isotopes) as a diagnostic/therapeutic radionuclide for BLC595. Labelling of antibodies with these radioisotopes are available in the literature and references are given below:

1) Technetium-99m:

Pimm M V, Gribben S J (1993) Radiolabelling antibodies for imaging and targeting. In: Tumour Immunobiology; A Practical Approach (Gallagher, Rees & Reynolds, eds) pp 209-223. Oxford University Press. (also for rhenium-188)

Mather S J & Ellison D (1990) Reduction mediated technetium-99m labelling of monoclonal antibodies. J. Nucl. Med 31: 692-697.

2) Rhenium-188:

Griffiths G L, Goldenberg D M, Diril H & Hansen H J (1994) Technetium-99 m, Rhenium-186 and Rhenium-188 direct-labeled antibodies. Cancer 73: 761-768.

Potential Usage of BLC595-based Radiopharmaceuticals Superficial Bladder Cancer: Intravesical Administration

The antibody can be utilised via the intravesical administration of BLC595 conjugated to radioactive isotopes to detect the presence of MUC1 mucin positive tumour cells within the confines of the bladder. Radionuclides include both ⁶⁷Cu and ^{99m}Tc for diagnostic purposes. Allied to the use of ^{99m}Tc is the isotope ¹⁸⁸Re, which has similar chemical characteristics to ^{99m}Tc but with a appropriate beta emission for cellular cytotoxicity and as such can be exploited in a therapeutic context. In a similar manner ⁶⁷Cu can be used in both a diagnostic and therapeutic scenario (it has both gamma and beta energy emission) although routine use of ⁶⁷Cu would be limited because it is not readily available widely.

Bladder Cancer: Invasive and Metastatic Disease

The same arguments apply for the use of BLC595 by systemic administration in the diagnosis and the treatment of metastatic bladder cancer. In human bladder cancer, we are not aware of the use of similar approaches using other radiolabelled anti-MUC1 mucin monoclonal antibodies. The humanised nature of BLC595 allow it to be administered repeatedly in multiple dosing regimens, whilst keeping the likelihood of human anti-mouse antibody (HAMA) response to a minimum. As a diagnostic and disease staging tool, preliminary data has shown that systemic use of the parent antibody C595 coupled to ¹¹¹In, ⁶⁷Cu, ^{99m}Tc and ¹⁸⁸Re would have the potential to be as useful as, if not better than, magnetic resonance imaging in instances where metastatic disease expresses MUC1. In the same way we would see therapeutic doses of radiolabelled antibody being utilised to treat patients of their disease.

Ovarian Cancer

Pre-clinical and clinical evaluation of the use of BLC595-based radioimmunoconjugates in the bladder cancer model should lead to their application in other diseases where MUC1 tumour expression is well characterised. This includes breast and ovarian carcinomas. In an ovarian study, we would use our reagents in diagnosis by their administration into the peritoneum. Because of the involvement of the hosts immune system in this cavity, the humanised antibody conjugate would offer the greatest chance of evading the HAMA response. Multiple administration for potential therapeutic effect could therefore be envisaged. Metastatic ovarian cancers may also be detected and treated in the same manner as metastatic bladder cancer using BLC595 conjugated to the aforesaid radionuclides.

Metastatic Breast Cancer

We could also see BLC595 finding a suitable role in the diagnosis and possible management of breast cancer. This again would involve systemic administration of the radioimmunoconjugate.

Current Phase I/II Trials

Our use of ⁶⁷Cu labelled C595 in a diagnostic context has been published. We now have approval from the Cancer Research Campaign (CRC) to begin a Phase I clinical trial in human bladder cancer using ⁶⁷Cu labelled C595 administered intravesically. Phase II trails using similar protocols should commence upon the completion of this study. This should ascertain the clinical utility of our radioimmunoconjugate (proof of principle) and should lead to similar trials being set up using ¹⁸⁸Re labelled C595, a more widely available radionuclide and therefore more commercially viable. Similar studies with radiolabelled BLC595 would follow after appropriate preclinical evaluation. The way forward into the systemic usage of this antibody would then be forged, so that experimentation on disseminated disease can progress. The use of appropriate higher does of this radioimmunoconjugate would see the use of this reagent in a potential therapeutic context.

The invention is not limited to the embodiments hereinbefore described which may be varied in construction and detail without departing from the spirit of the invention.

### REFERENCES

Bendig M M, Jones S T (1996) Rodent to human antibodies by CDR grafting. IN: McCafferty J, Hoogenboom, H R, Chiswell D J (eds) Antibody engineering—a practical approach. NEW YORK: Oxford University Press.

Chothia C, Novotny J, Bruccoleri R, Karplus M (1985) Domain association in immunoglobulin molecules—the packing of variable domains. J Mol Biol 186:651-663.

Foote J, Winter G (1992) Antibody framework residues affecting the conformation of the hypervariable loops. J Mol Biol 224:487499

Higuchi R, Krummel B, Saiki R K (1988) A general method of in vitro preparation and specific mutagenesis of DNA fragments: Study of protein and DNA interactions. Nucleic Acids Res 16:7351-7367

Kabat E A, Wut T T, Perry H M, Gottesman K S, Foeller C (1991) Sequences of proteins of immunological interest. 5^{th} edition. BETHESDA: US Department of Health and Human Services.

Kozak M (1987) At least six nucleotides preceding the AUG initiator codon enhance translation in mammalian cells. J Mol Biol 196:947-950

Padlan E A (1994) Anatomy of the antibody molecule. Mol Immunol 31(3):169-217.

1. A humanized antibody capable of binding to a MUC1 mucin antigen comprising a light chain and a heavy chain, the variable region of the light chain (V_{L}) comprising an amino acid sequence which is substantially homologous with the sequence of FIG. 1A, the variable region of the heavy chain (V_{H}) comprising an amino acid sequence which is substantially homologous with the sequence of FIG. 1B, wherein the amino acid residue at position 46 on V_{L} is backmutated to arginine, and wherein the amino acid residue at position 47 on V_{H} is backmutated to leucine.

2. A humanized antibody as claimed in claim 1 in which the amino acid residue at position 4 of V_{L} is backmutated to leucine.

3. A humanized antibody as claimed in claim 2 in which the amino acid residue at position 1 of V_{L} is backmutated to glutamin.

4. A humanized antibody as claimed in claim 3 in which the amino acid residue at position 47 on V_{L} is backmutated to tryptophan.

5. A humanized antibody as claimed in claim 4 in which the amino acid residue at position 3 on V_{L} is backmutated to valine.

6. A humanized antibody as claimed in claim 5 in which the amino acid residues at positions 40 and 70 on V_{L} are backmutated to serine.

7. A humanized antibody as claimed in claim 1 in which the amino acid residue at position 47 on V_{L} is backmutated to tryptoptian.

8. A humanized antibody as claimed in claim 2 in which the amino acid residue at position 3 on V_{L} is backmutated to valine.

9. A humanized antibody as claimed in claim 3 in which the amino acid residue at position 47 on V_{L} is backmutated to tryptophan.

10. A humanised humanized antibody as claimed in any preceding claim in which the amino acid residue at position 42 on V_{H} is backmutated to aspartic acid.

11. A humanized antibody as claimed in claim 10 in which the amino acid residue at position 16 on V_{H} is backmutated to glycine.

12. A humanized antibody as claimed in claim 10 in which the amino acid residue at position 44 on V_{H} is backmutated to arginine.

13. A humanized antibody as claimed in claim 10 in which the amino acid residue at position 11 on V_{H} is backmutated to leucine.

14. A humanized antibody as claimed in claim 10 in which the amino acid residue at position 19 on V_{H} is backmutated to lysine.

15. A humanized antibody as claimed in claim 1 in which the amino acid residues at positions 11, 16 and 19 on V_{H} are backmutated to leucine, glycine and lysine respectively.

16. A humanized antibody as claimed in claim 1 in which the amino acid residues at positions 40, 82a and 108 on V_{H} are backmutated to threonine, serine and threonine respectively.

17. A humanized antibody as claimed in claim 1 in which the amino acid residue at position 74 on V_{H} backmutated to alanine.

18. A humanized antibody as claimed in claim 1 in which the amino acid residue at position 89 on V_{H} is backmutated to methionine.

19. A humanized antibody as claimed in claim 1 in which the amino acid residues at positions 108 and 109 on V_{H} are backmutated to threonine and leucine respectively.

20. A humanized antibody as claimed in claim 1 in which the amino acid residue at positions 83 and 84 on V_{H} are backmutated to lysine and serine respectively.

21. A humanized antibody as claimed in claim 1 in which the V_{L} domain comprises a framework region (FR) and complementarity determining regions (CDR), wherein the FR region comprises the Bence Jones protein REI, and wherein the CDR are obtained from C595 antibody.

22. A humanized antibody as claimed in claim 1 in which the V_{H} domain comprises a framework region (FR) and complementarity determining regions (CDR), wherein the FR region comprises the meyloma protein HIL, and wherein the CDR are obtained from C595 antibody.

23. A humanized antibody as claimed in claim 1 conjugated to a radioactive isotope.

24. A humanized antibody as claimed in claim 23 in which the radioactive isotope is selected from the group of Technetium-99m, Rhenium-188, Copper-67 and Indium-111.

25. Use of a humanized antibody as claimed in claim 1 in the diagnosis and/or treatment of cancer.

26. Use of a humanized antibody as claimed in claim 1 in the intravesical diagnosis and/or therapy of bladder tumour and/or bladder cancer.

27. Use of a humanized antibody as claimed in claim 1 in the intravenous diagnosis, staging and/or therapy of metastatic bladder cancer.

28. Use of a humanized antibody as claimed in claim 1 in the intravenous diagnosis and/or therapy of metastatic cancers expressing the MUC1 mucin antigen, especially bladder, breast and ovarian cancers.

29. A variable light chain domain V_{L} for a humanized antibody according to claim 1 comprising an amino acid sequence which has a sufficient degree of homology with the sequence of FIG. 1A to allow binding to the MUC1 mucin antigen when one of the backmutation combinations given in Table 2A is included.

30. A variable heavy chain domain V_{H} for a humanised humanized antibody according to claim 1 and comprising an amino acid sequence which has a sufficient degree of homology with the sequence of FIG. 1B to allow binding to the MUC1 mucin antigen when one of the backmutation combinations given in Table 2B is included.

31. Use of at least one of the V_{L} domain of claim 29 and the V_{H} domain of claim 30 in the formation of a humanized antibody and/or an antibody binding fragment which is capable of binding to the MUC1 mucin antigen.

32. A method for the treatment or diagnosis of cancer, comprising administering an effective amount of a humanized antibody according to claim 1 to a patient.

33. A humanized antibody according to claim 1 for use in the manufacture of a medicament for the treatment or diagnosis of cancer.

34. A nucleic acid sequence which codes for a humanized antibody of claim 1.


SEQUENCE LISTING (36 sequences)

SEQ ID NO: 1 - protein, 106 aa, Artificial Sequence
  Description of Artificial Sequence Humanised Antibody BLC595a
DIQMTQSPSSLSASVGDRVTITCSASSSVSYMHWYQQKPGKAPKLLIYDTSKLASGVPSRFSGSGSGTDYTFTISSLQPEDIATYYCQQWSSNPPTFGQGTKLQIK

SEQ ID NO: 2 - protein, 120 aa, Artificial Sequence
  Description of Artificial Sequence Humanised Antibody BLC595a
EVQLVESGGGVVQPGRSLRLSCAASGFTFSSYGMSWVRQAPGKGLEWVATINSNGGSTYYPDSVKGRFTISRDNSKNTLYLQMNSLRTEDTAVYYCARDRDGYDEGFDYWGQGTLVTVSS

SEQ ID NO: 3 - DNA, 429 bp, Artificial Sequence
  Description of Artificial Sequence Encoding sequence for BLC595a
aatcgatacgctccaagcttgccgccaccatgggatggagctgtatcatcctcttcttggtagcaacagctacaggtgtccactccgatattcagatgacccagtctccatcctccctgtctgcatctgtaggagatagagtcaccatcacctgcagtgccagttcaagtgtaagttatatgcactggtaccagcagaaaccaggcaaagctcctaaactcctgatctatgacacatccaaactggcttctggagtcccatcaaggttcagtggcagtgggtctgggacagattacactttcaccatcagcagcctgcagcctgaagatattgcaacttattactgccagcagtggagtagtaacccgcccacgttcggtcaagggaccaagttgcagatcaaacgtaagtggatccaattagccgagt

SEQ ID NO: 4 - DNA, 80 bp, Artificial Sequence
  Description of Artificial Sequence Oligonucleotide
aatcgatacgctccaagcttgccgccaccatgggatggagctgtatcatcctcttcttggtagcaacagctacaggtgtc

SEQ ID NO: 5 - DNA, 78 bp, Artificial Sequence
  Description of Artificial Sequence Oligonucleotide
tcctacagatgcagacagggaggatggagactgggtcatctgaatatcggagtggacacctgtagctgttgctaccaa

SEQ ID NO: 6 - DNA, 78 bp, Artificial Sequence
  Description of Artificial Sequence Oligonucleotide
tcctccctgtctgcatctgtaggagatagagtcaccatcacctgcagtgccagttcaagtgtaagttatatgcactgg

SEQ ID NO: 7 - DNA, 78 bp, Artificial Sequence
  Description of Artificial Sequence Oligonucleotide
tttggatgtgtcatagatcaggagtttaggagctttgcctggtttctgctggtaccagtgcatataacttacacttga

SEQ ID NO: 8 - DNA, 78 bp, Artificial Sequence
  Description of Artificial Sequence Oligonucleotide
aaagctcctaaactcctgatctatgacacatccaaactggcttctggagtcccatcaaggttcagtggcagtgggtct

SEQ ID NO: 9 - DNA, 78 bp, Artificial Sequence
  Description of Artificial Sequence Oligonucleotide
agttgcaatatcttcaggctgcaggctgctgatggtgaaagtgtaatctgtcccagacccactgccactgaaccttga

SEQ ID NO: 10 - DNA, 78 bp, Artificial Sequence
  Description of Artificial Sequence Oligonucleotide
ctgcagcctgaagatattgcaacttattactgccagcagtggagtagtaacccgcccacgttcggtcaagggaccaag

SEQ ID NO: 11 - DNA, 61 bp, Artificial Sequence
  Description of Artificial Sequence Oligonucleotide
actcggctaattggatccacttacgtttgatctgcaacttggtcccttgaccgaacgtggg

SEQ ID NO: 12 - DNA, 24 bp, Artificial Sequence
  Description of Artificial Sequence Oligonucleotide
tcgatacgctccaagcttgccgcc

SEQ ID NO: 13 - DNA, 24 bp, Artificial Sequence
  Description of Artificial Sequence Oligonucleotide
gtgtcatagatcaggagtttagga

SEQ ID NO: 14 - DNA, 24 bp, Artificial Sequence
  Description of Artificial Sequence Oligonucleotide
cctaaactcctgatctatgacaca

SEQ ID NO: 15 - DNA, 24 bp, Artificial Sequence
  Description of Artificial Sequence Oligonucleotide
ctcggctaattggatccacttacg

SEQ ID NO: 16 - DNA, 24 bp, Artificial Sequence
  Description of Artificial Sequence Oligonucleotide
cgctccaagcttgccgccaccatg

SEQ ID NO: 17 - DNA, 24 bp, Artificial Sequence
  Description of Artificial Sequence Oligonucleotide
taattggatccacttacgtttgat

SEQ ID NO: 18 - DNA, 471 bp, Artificial Sequence
  Description of Artificial Sequence Encoding sequence for BLC595a
aatcgatacgctccaagcttgccgccaccatggagtttgggctgagctggctttttcttgtggctattttaaaaggtgtccagtgtgaggtgcagctggtggagtctggaggaggcgtggtgcagcctgggcgttcactgagactctcctgcgcagcttctggattcaccttcagtagctacggtatgagctgggtgcgccaggctccaggaaagggccttgagtgggtcgcaaccattaatagtaatggtggtagcacttactacccagactctgtgaagggccgattcacaatctccagagacaattccaagaacacactgtacctgcagatgaacagcctgagaactgaggacacagccgtctattactgtgcaagagatagggatggttacgatgaaggttttgactactggggccaagggaccctggtcaccgtctcctcaggtaagtggatccaattagccgagt

SEQ ID NO: 19 - DNA, 83 bp, Artificial Sequence
  Description of Artificial Sequence Oligonucleotide
aatcgatacgctccaagcttgccgccaccatggagtttgggctgagctggctttttcttgtggctattttaaaaggtgtccag

SEQ ID NO: 20 - DNA, 81 bp, Artificial Sequence
  Description of Artificial Sequence Oligonucleotide
cagtgaacgcccaggctgcaccacgcctcctccagactccaccagctgcacctcacactggacaccttttaaaatagccac

SEQ ID NO: 21 - DNA, 81 bp, Artificial Sequence
  Description of Artificial Sequence Oligonucleotide
gtggtgcagcctgggcgttcactgagactctcctgcgcagcttctggattcaccttcagtagctacggtatgagctgggtg

SEQ ID NO: 22 - DNA, 81 bp, Artificial Sequence
  Description of Artificial Sequence Oligonucleotide
accaccattactattaatggttgcgacccactcaaggccctttcctggagcctggcgcacccagctcataccgtagctact

SEQ ID NO: 23 - DNA, 81 bp, Artificial Sequence
  Description of Artificial Sequence Oligonucleotide
cttgagtgggtcgcaaccattaatagtaatggtggtagcacttactacccagactctgtgaagggccgattcacaatctcc

SEQ ID NO: 24 - DNA, 81 bp, Artificial Sequence
  Description of Artificial Sequence Oligonucleotide
gtcctcagttctcaggctgttcatctgcaggtacagtgtgttcttggaattgtctctggagattgtgaatcggcccttcac

SEQ ID NO: 25 - DNA, 81 bp, Artificial Sequence
  Description of Artificial Sequence Oligonucleotide
atgaacagcctgagaactgaggacacagccgtctattactgtgcaagagatagggatggttacgatgaaggttttgactac

SEQ ID NO: 26 - DNA, 82 bp, Artificial Sequence
  Description of Artificial Sequence Oligonucleotide
actcggctaattggatccacttacctgaggagacggtgaccagggtcccttggccccagtagtcaaaaccttcatcgtaacc

SEQ ID NO: 27 - DNA, 24 bp, Artificial Sequence
  Description of Artificial Sequence Oligonucleotide
tcgatacgctccaagcttgccgcc

SEQ ID NO: 28 - DNA, 24 bp, Artificial Sequence
  Description of Artificial Sequence Oligonucleotide
tactattaatggttgcgacccact

SEQ ID NO: 29 - DNA, 24 bp, Artificial Sequence
  Description of Artificial Sequence Oligonucleotide
gagtgggtcgcaaccattaatagt

SEQ ID NO: 30 - DNA, 24 bp, Artificial Sequence
  Description of Artificial Sequence Oligonucleotide
ctcggctaattggatccacttacc

SEQ ID NO: 31 - DNA, 23 bp, Artificial Sequence
  Description of Artificial Sequence Oligonucleotide
cgctccaagcttgccgccaccat

SEQ ID NO: 32 - DNA, 24 bp, Artificial Sequence
  Description of Artificial Sequence Oligonucleotide
taattggatccacttacctgagga

SEQ ID NO: 33 - protein, 106 aa, Artificial Sequence
  Description of Artificial Sequence Humanised antibody BLC595
QIQLTQSPSSLSASVGDRVTITCSASSSVSYMHWYQQKPGKAPKRWIYDTSKLASGVPSRFSGSGSGTDYTFTISSLQPEDIATYYCQQWSSNPPTFGQGTKLQIK

SEQ ID NO: 34 - protein, 120 aa, Artificial Sequence
  Description of Artificial Sequence Humanised antibody BLC595
EVQLVESGGGVVQPGGSLRLSCAASGFTFSSYGMSWVRQAPDKGLELVATINSNGGSTYYPDSVKGRFTISRDNSKNTLYLQMNSLRTEDTAVYYCARDRDGYDEGFDYWGQGTLVTVSS

SEQ ID NO: 35 - DNA, 429 bp, Artificial Sequence
  Description of Artificial Sequence Encoding sequence for BLC595a
actcggctaattggatccacttacgtttgatctgcaacttggtcccttgaccgaacgtgggcgggttactactccactgctggcagtaataagttgcaatatcttcaggctgcaggctgctgatggtgaaagtgtaatctgtcccagacccactgccactgaaccttgatgggactccagaagccagtttggatgtgtcatagatcaggagtttaggagctttgcctggtttctgctggtaccagtgcatataacttacacttgaactggcactgcaggtgatggtgactctatctcctacagatgcagacagggaggatggagactgggtcatctgaatatcggagtggacacctgtagctgttgctaccaagaagaggatgatacagctccatcccatggtggcggcaagcttggagcgtatcgatt

SEQ ID NO: 36 - DNA, 471 bp, Artificial Sequence
  Description of Artificial Sequence Encoding sequence for BLC595a
actcggctaattggatccacttacctgaggagacggtgaccagggtcccttggccccagtagtcaaaaccttcatcgtaaccatccctatctcttgcacagtaatagacggctgtgtcctcagttctcaggctgttcatctgcaggtacagtgtgttcttggaattgtctctggagattgtgaatcggcccttcacagagtctgggtagtaagtgctaccaccattactattaatggttgcgacccactcaaggccctttcctggagcctggcgcacccagctcataccgtagctactgaaggtgaatccagaagctgcgcaggagagtctcagtgaacgcccaggctgcaccacgcctcctccagactccaccagctgcacctcacactggacaccttttaaaatagccacaagaaaaagccagctcagcccaaactccatggtggcggcaagcttggagcgtatcgatt